(19) United States
(12) Patent Application Publication
(10) Pub. No.: US 2024/0280448 A1
(43) Pub. Date: Aug. 22, 2024

(54) CASSETTE FOR HOLDING AND ORIENTING TISSUE SAMPLE

(71) Applicant: LEICA BIOSYSTEMS NUSSLOCH GMBH, Nussloch (DE)

(72) Inventors: Changhong WU, Shanghai (CN); Chun JIN, Shanghai (CN); Jue CHEN, Shanghai (CN)

(21) Appl. No.: 18/570,294

(22) PCT Filed: Jun. 18, 2021
(86) PCT No.: PCT/CN2021/100896

§ 371 Date: Dec. 14, 2023

(51) Int. Cl. G01N 1/36; A61B 10/00

(52) U.S. Cl. CPC G01N 1/36; A61B 10/0096

(57) ABSTRACT

A cassette, including: a frame defining an accommodating chamber; a retaining assembly coupled to the frame and movably arranged in the accommodating chamber; and a base detachably coupled to the retaining assembly and configured to be moved along with the retaining assembly, the retaining assembly being configured to retain the tissue sample in the base. The retaining assembly and the base are movable between a first position where the base is received in the accommodating chamber and a second position where the base is protruded out of the accommodating chamber, and the base is configured to be detached from the retaining assembly when the retaining assembly is in the second position.

### FIELD

The present disclosure relates to a field of tissue processing and embedding, and more particularly to a cassette for holding and orienting a tissue sample.

### BACKGROUND

A biopsy is the removal of a tissue sample to examine tissue for signs of cancer or other disorders. Tissue samples are obtained in a variety of ways using various medical procedures involving a variety of the sample collection devices. For example, biopsies may be open (surgically removing tissue) or percutaneous (e.g. by fine needle aspiration, core needle biopsy or vacuum assisted biopsy).

After the tissue sample is collected, the tissue sample is analyzed at a lab (e.g. a pathology lab, biomedical lab, etc.) that is set up to perform the appropriate tests (such as histological analysis). In order to properly process the tissue sample, a series of steps may be performed, including: grossing of the tissue sample by cutting the tissue sample to the proper size for analysis; processing of the tissue sample to immobilize molecular components and/or prevent degradation, generally including fixation, dehydration, and clearing of the tissue sample; embedding the tissue sample in an embedding material, such as paraffin wax; sectioning the embedded tissue sample by using, for example, a microtome.

Currently, after the processing of the tissue sample, a user needs to manually open the cassette and transfer the tissue sample to an embedding mold, and also needs to manually orientate and embed the tissue sample, which will reduce the user's working efficiency.

CA2566024 relates to a cassette for transporting a tissue, including a first flat reference porous structure for supporting the tissue, and a second porous structure having a compression resistance. The first flat reference porous structure and the second porous structure can be positioned into an abutting, contacting or non-contacting position for securing the tissue therebetween.

CN104853843B relates to a sectionable tissue sample support structure, including a gel compound formed into a self-supporting geometric shape for retention and orientation of at least one tissue sample during a histopathology process which includes processing, embedding and microtome slicing of the tissue sample.

EP1842044B1 relates to a system for use in processing a tissue specimen for histological examination, including a tissue specimen positioning member, a carrier member for receiving the positioning member with tissue specimen thereon and for subsequently mounting in a microtome the tissue specimen as processed to be associated with a fixing medium, and a base member having a lower recessed chamber sized to receive the tissue specimen and a method for preparing a tissue specimen for histological examination.

U.S. Pat. No. 8,383,067B2 relates to a histologic tissue sample support device, which includes a tissue support formed of material that can be successfully sectioned in a microtome and is resistant to degradation from solvents and chemicals used to fix, process and stain tissue. A resilient cellular material is coupled to the tissue support and is configured to engage and retain tissue in place during processing and embedding. The resilient cellular material is also capable of successful sectioning in the microtome and porous to allow infiltration of the solvents and chemicals used to fix, process and stain tissue, and of embedding material used to embed the tissue while the tissue is retained by the resilient cellular material.

Although some of the above devices or methods allow the tissue sample to be embedded automatically, the user needs to section the cassette material, which will low down the microtome blade's life and decrease the sectioning efficiency.

### SUMMARY

Embodiments of the present disclosure seek to solve at least one of the problems existing in the prior art to at least some extent, and thus provide a cassette having a detachable base.

According to embodiments of the present disclosure, there is provided a cassette. The cassette includes: a frame defining an accommodating chamber; a retaining assembly coupled to the frame and movably arranged in the accommodating chamber; and a base detachably coupled to the retaining assembly for receiving the tissue sample and configured to be moved along with the retaining assembly, the retaining assembly being configured to retain the tissue sample in the base. The retaining assembly and the base are movable between a first position where the base is received in the accommodating chamber and a second position where the base is protruded out of the accommodating chamber, and the base is configured to be detached from the retaining assembly when the retaining assembly is in the second position.

In the cassette according to embodiments of the present disclosure, the base is coupled to the retaining assembly, and the retaining assembly is configured to retain the tissue sample in the base. Thus, the tissue sample can be held and oriented between the base and the retaining assembly. Furthermore, the base can be moved along with the retaining assembly between the first position and the second position. Thus, the cassette can be used in both tissue processing and embedding, and the orientation of the tissue sample is not necessary to change. Moreover, the base can be detached from the retaining assembly when the retaining assembly is in the second position during the tissue embedding, so as to finally obtain a wax block without the base embedded. Thus, the embedded tissue sample can be sectioned without cutting the base.

In some embodiments, the retaining assembly includes: an intermediate member arranged in the accommodating chamber and having a through hole in the middle, the intermediate member being movable between the first position and the second position; a cover rotatably connected to the intermediate member and configured to be closed to cover the through hole; and a retaining member connected to the cover and configured to pass through the through hole to retain the tissue sample in the base.

In some embodiments, the base is detachably connected to the intermediate member and configured to be moved along with the intermediate member between the first position and the second position.

In some embodiments, the intermediate member has a first surface and a second surface facing away from each other, the cover is rotatably connected to the first surface, and the base is detachably connected to the second surface.

In some embodiments, the intermediate member includes a first engaging member and a first snap protruded from the first surface, and a second snap protruded from the second surface; the cover has a second engaging member rotatably engaged with the first engaging member, and a first snapping hole into which the first snap is configured to be snapped; and the base has a second snapping hole into which the second snap is configured to be snapped.

In some embodiments, two first snaps are provided at two opposite edges of the through hole of the intermediate member, and the first engaging member is arranged between an edge of the intermediate member and one of the first snaps; and two first snapping holes are formed in the cover and correspond to the two first snaps, respectively.

In some embodiments, four second snaps are provided at two opposite edges of the through hole of the intermediate member, two of the four second snaps are arranged at one of the two opposite edges of the through hole, and the other two of the four second snaps are arranged at the other one of the two opposite edges of the through hole; and four second snapping holes are formed in the base and correspond to the four second snaps, respectively.

In some embodiments, the base includes a receptacle having an opening and a flange connected to the receptacle and surrounding the opening of the receptacle, and the second snapping hole is formed in the flange of the base.

In some embodiments, the receptacle includes a bottom and a side wall extending from an edge of the bottom, and the bottom defines a plurality of through holes.

In some embodiments, the cover includes a body and a fixing member arranged to a surface of the body configured to be in contact with the intermediate member, and the retaining member is fixed to the body by the fixing member.

In some embodiments, the fixing member includes a plurality of projections extending from the surface of the body, and the retaining member is surrounded and fixed by the plurality of projections.

In some embodiments, the body defines a plurality of through holes overlapping at least part of the retaining member.

In some embodiments, the intermediate member includes a rim extending outwards from an outer side wall of the intermediate member, the frame includes a first limiting member on an inner side wall of the frame and adjacent to a first edge of the inner side wall of the frame, and the first limiting member is configured to be fitted with the rim to limit the retaining assembly in the first position.

In some embodiments, the first limiting member includes a first protrusion extending inwards from the inner side wall of the frame, and the first protrusion is configured to support a surface of the rim of the intermediate member facing the base when the retaining assembly is in the first position.

In some embodiments, the surface of the rim of the intermediate member facing the base defines a first groove, and the first protrusion is configured to be fitted in the first groove when the retaining assembly is in the first position.

In some embodiments, the first limiting member further includes a second protrusion extending inwards from the inner side wall of the frame, the first protrusion is spaced apart from the second protrusion in a direction from the first position to the second direction, and the second protrusion is configured to abut against a surface of the rim of the intermediate member facing away from the base when the retaining assembly is in the first position.

In some embodiments, the surface of the rim of the intermediate member facing away from the base defines a second groove, and the second protrusion is configured to be fitted in the second groove when the retaining assembly is in the first position.

In some embodiments, the frame includes a second limiting member on the inner side wall of the frame and adjacent to a second edge of the inner side wall of the frame, the second edge of the inner side wall of the frame is opposite to the first edge of the inner side wall of the frame in a direction from the first position to the second position, and the second limiting member is configured to be fitted with the rim to limit the retaining assembly in the second position.

In some embodiments, the second limiting member includes a step extending inwards from the second edge of the inner side wall of the frame, and the step is configured to support a surface of the rim facing the base when the retaining assembly is in the second position.

In some embodiments, the second limiting member includes a third protrusion extending inwards from the inner side wall of the frame, the step is spaced part from the third protrusion in the direction from the first position to the second direction, and the third protrusion is configured to abut against a surface of the rim facing away from the base when the retaining assembly is in the second position.

Additional aspects and advantages of embodiments of present disclosure will be given in part in the following descriptions, become apparent in part from the following descriptions, or be learned from the practice of the embodiments of the present disclosure.

### BRIEF DESCRIPTION OF THE DRAWINGS

These and other aspects and advantages of embodiments of the present disclosure will become apparent and more readily appreciated from the following descriptions made with reference the accompanying drawings.

FIG. 1 is a perspective view of a cassette with a cover being opened according to an embodiment of the present disclosure.

FIG. 2 is a perspective view of a cassette in a first position according to an embodiment of the present disclosure.

FIG. 3 is another perspective view of a cassette in a first position according to an embodiment of the present disclosure from another angle.

FIG. 4 is a perspective view of a cassette in a second position according to an embodiment of the present disclosure.

FIG. 5 is another perspective view of a cassette in a second position according to an embodiment of the present disclosure from another angle.

FIG. 6 is a perspective view of a frame of a cassette according to an embodiment of the present disclosure.

FIG. 7 is another perspective view of a frame of a cassette according to an embodiment of the present disclosure from another angle.

FIG. 8 is a perspective view of an intermediate member of a cassette according to an embodiment of the present disclosure.

FIG. 9 is another perspective view of an intermediate member of a cassette according to an embodiment of the present disclosure.

FIG. 10 is a perspective view of a cover of a cassette according to an embodiment of the present disclosure.

FIG. 11 is another perspective view of a cover of a cassette according to an embodiment of the present disclosure.

FIG. 12 is a perspective view of a base of a cassette according to an embodiment of the present disclosure.

FIG. 13 is another perspective view of a base of a cassette according to an embodiment of the present disclosure.

### DETAILED DESCRIPTION

Reference will be made in detail to embodiments of the present disclosure. The embodiments described herein with reference to drawings are explanatory, illustrative, and used to generally understand the present disclosure. The embodiments shall not be construed to limit the present disclosure. The same or similar elements and the elements having same or similar functions are denoted by like reference numerals throughout the descriptions.

In the specification, Unless specified or limited otherwise, relative terms such as “central”. “longitudinal”, “lateral”, “front”, “rear”, “right”, “left”, “inner”, “outer”, “lower”, “upper”, “horizontal”, “vertical”, “above”, “below”, “up”, “top”, “bottom” as well as derivative thereof (e.g., “horizontally”, “downwardly”, “upwardly”, etc.) should be construed to refer to the orientation as then described or as shown in the drawings under discussion. These relative terms are for convenience of description and do not require that the present disclosure be constructed or operated in a particular orientation.

Terms concerning attachments, coupling and the like, such as “connected” and “interconnected”, refer to a relationship wherein structures are secured or attached to one another either directly or indirectly through intervening structures, as well as both movable or rigid attachments or relationships, unless expressly described otherwise.

Unless specified or limited otherwise, the terms “mounted,” “connected.” “supported.” and “coupled” and variations thereof are used broadly and encompass both direct and indirect mountings, connections, supports, and couplings. Further, “connected” and “coupled” are not restricted to physical or mechanical connections or couplings.

Also, it is to be understood that phraseology and terminology used herein with reference to device or element orientation (such as, for example, terms like “central,” “upper,” “lower,” “front.” “rear,” and the like) are only used to simplify description of the present disclosure, and do not alone indicate or imply that the device or element referred to must have a particular orientation.

In addition, terms such as “first” and “second” are used herein for purposes of description and are not intended to indicate or imply relative importance or significance.

Embodiments of the present disclosure provide a cassette 1000 for holding and orientating a tissue sample 400. The cassette 1000 includes a frame 100, a retaining assembly 200 and a base 300. The frame 100 defines an accommodating chamber 101. The retaining assembly 200 is coupled to the frame 100 and movably arranged in the accommodating chamber 101. The base 300 is detachably coupled to the retaining assembly 200 for receiving the tissue sample 400 and configured to be moved along with the retaining assembly 200. The retaining assembly 200 is configured to retain the tissue sample 400 in the base 300. The retaining assembly 200 and the base 300 are movable between a first position where the base 300 is received in the accommodating chamber 101 and a second position where the base 300 is protruded out of the accommodating chamber 101. That is, the retaining assembly 200 and the base 300 can move synchronously between the first position and the second position. Further, the base 300 is configured to be detached from the retaining assembly 200 when the retaining assembly 200 in the second position.

In the cassette 1000 according to embodiments of the present disclosure, the base 300 is coupled to the retaining assembly 200, and the retaining assembly 200 is configured to retain the tissue sample in the base 300. Thus, the tissue sample 400 can be held and oriented between the base 300 and the retaining assembly 200. Furthermore, the base 300 can be moved along with the retaining assembly 200 between the first position and the second position. Thus, the cassette 1000 can be used in both tissue processing and embedding, and the orientation of the tissue sample 400 is not necessary to change. Moreover, the base 300 can be detached from the retaining assembly 200 when the retaining assembly 200 in the second position during the tissue embedding, so as to finally obtain a wax block without the base 300 embedded. Thus, the embedded tissue sample 400 can be sectioned without cutting the base 300.

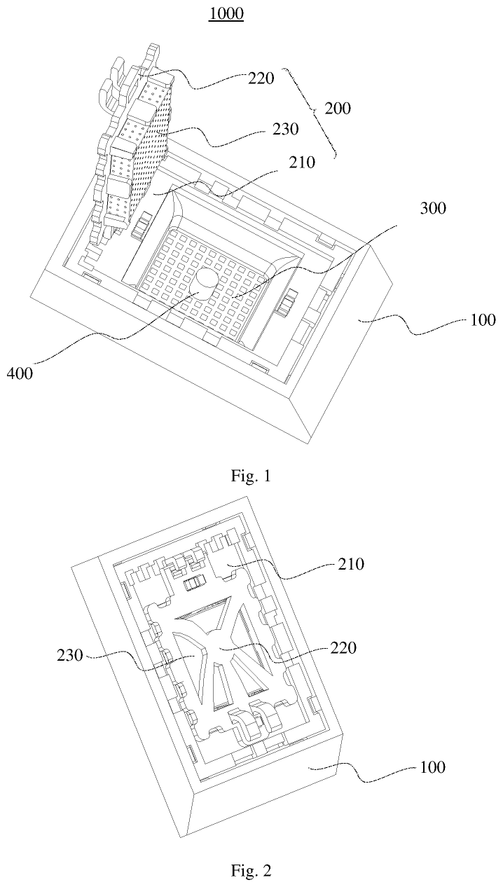

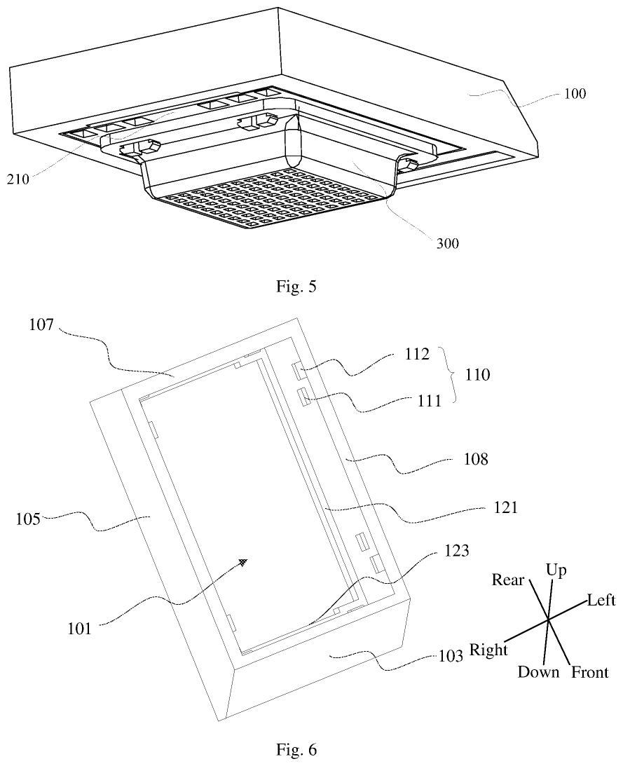

As illustrated in FIGS. 1-5 and 8-9, the retaining assembly 200 includes an intermediate member 210, a cover 220, and a retaining member 230. The intermediate member 210 is arranged in the accommodating chamber 101 and has a through hole 218 in the middle, and the intermediate member 210 is movable between the first position and the second position. In some embodiments, the intermediate member 210 may be an intermediate bracket having a square or rectangular shape, and a middle portion in a square or rectangular shape of the intermediate bracket is removed. The cover 220 is rotatably connected to the intermediate member 210 and configured to be closed to cover the through hole 218. Specifically, the cover 220 can be rotated to be opened so as to expose the through hole 218 of the intermediate member 210, and thus the tissue sample 400 can be put into the base 300. Further, the cove 220 can also be rotated to be closed after the tissue sample 400 is placed in the base 300, so as to cover the through hole 218 of the intermediate member 210, and allow the retaining member 230 to retain and orient the tissue sample 400. Specifically, the retaining member 230 is connected to the cover 220 and configured to pass through the through hole 218 of the intermediate member 210 to retain the tissue sample in the base 300. That is, when the cover 220 is rotated to be closed after the tissue sample is placed in the base 300, the retaining member 230 will move along with the cover 220 and pass through the through hole 218 of the intermediate member 210, so as to retain the tissue sample in the base 300. Thus, the tissue sample 400 can be held and oriented in the base 300.

Further, the base 300 is detachably connected to the intermediate member 210 and configured to be moved along with the intermediate member 210 between the first position and the second position. That is, the intermediate 201 and the base 300 can move synchronously between the first position and the second position, such that the cassette 1000 can be used for both the tissue processing when the base 300 is in the first position and for the tissue embedding when the base 300 is in the second position.

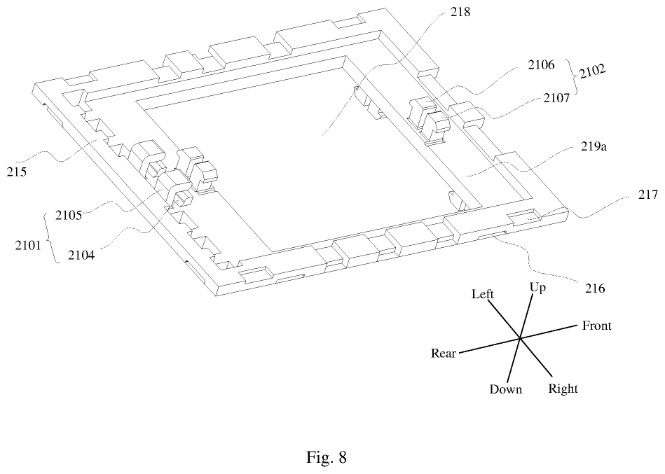

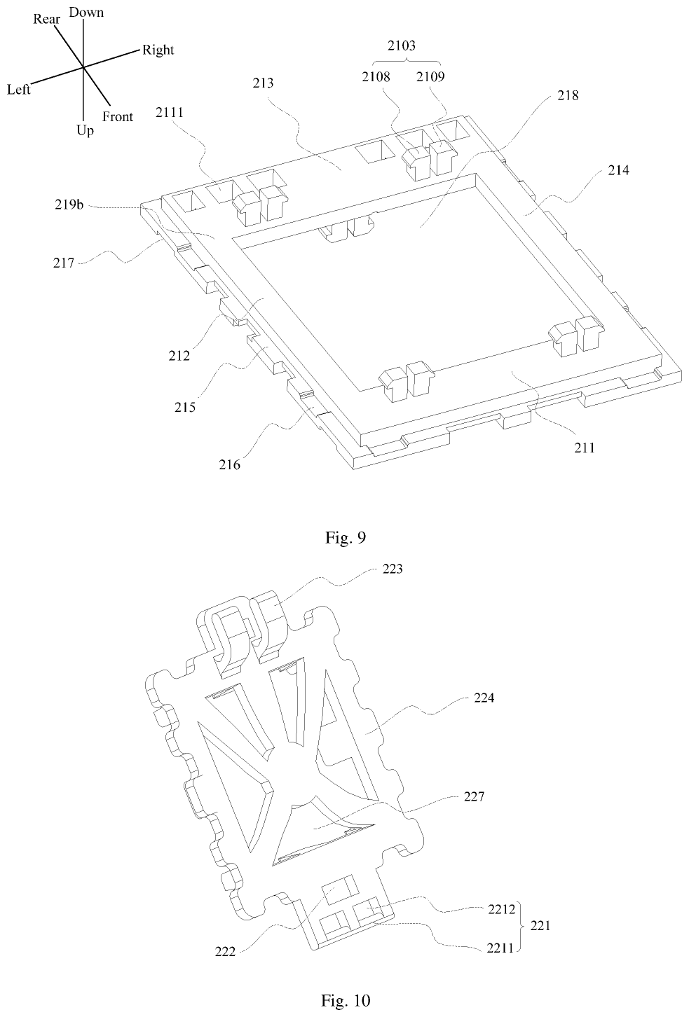

In some embodiments, as illustrated in FIGS. 8 and 9, the intermediate member 210 has a first surface 219a and a second surface 219b facing away from each other. The cover 220 is rotatably connected to the first surface 219a, and the base 300 is detachably connected to the second surface 219b.

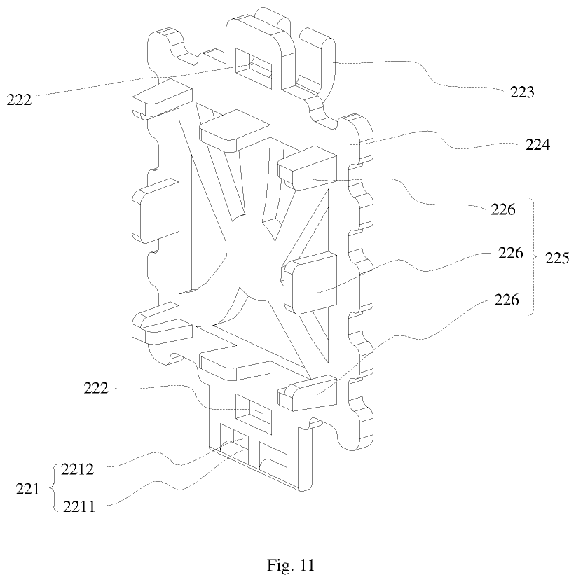

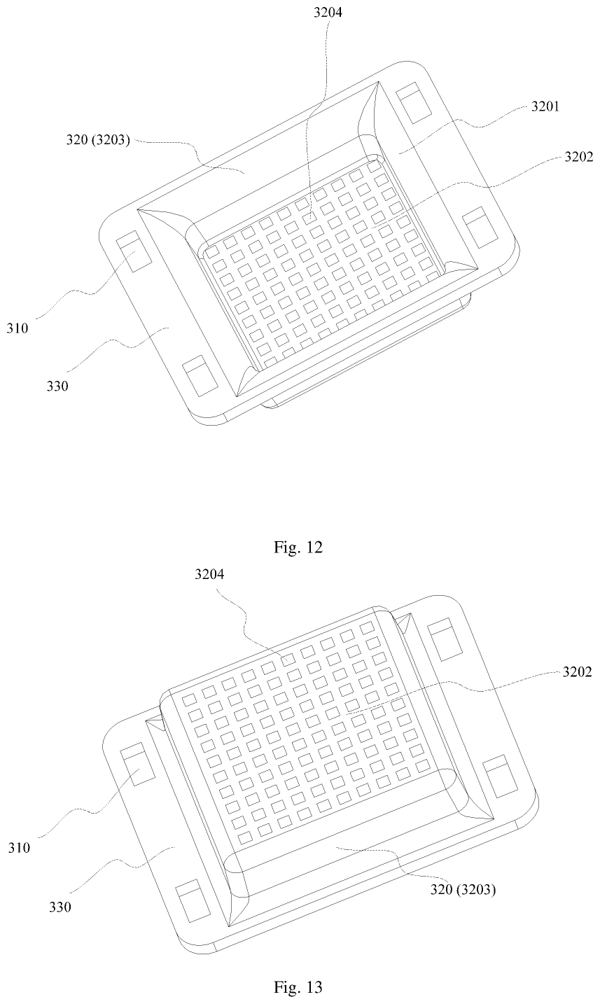

Specifically, as further illustrated in FIGS. 8 and 9, the intermediate member 210 includes a first engaging member 2101 and a first snap 2102 protruded from the first surface 219a, and a second snap 2103 protruded from the second surface 219b. As illustrated in FIGS. 10 and 11 in combination with FIGS. 1-5, the cover 220 has a second engaging member 221 rotatably engaged with the first engaging member 2101, and a first snapping hole 222 into which the first snap 221 is configured to be snapped. As illustrated in FIGS. 12 and 13 in combination with FIGS. 1-5, the base 300 has a second snapping hole 310 into which the second snap 2103 is configured to be snapped.

Thus, the cover 220 is rotatably connected to the intermediate member 210 through the rotatable engagement between the first engaging member 2101 and the second engaging member 221. Specifically, the second engaging member 221 may include an engaging shaft 2211 arranged at an edge of the cover 220 and an engaging opening 2212 adjacent to the engaging shaft 2211. Accordingly, the first engaging member 2101 may include a boss 2104 and a stopping member 2105 protruded from the first surface 219a of the intermediate member 210. The boss 2104 and the stopping member 2105 are spaced from each other, and a free end of the stopping member 2105 is bent towards the boss 2104, so that a space is defined by the stopping member 2105 and the boss 2104, and a gap is defined between the free end of the stopping member 2105 and the boss 2104. Thus, the engaging shaft 2211 can be placed into the space defined by the boss 2104 and the stopping member 2105 through the gap, and hence fitted between the boss 2104 and the stopping member 2105. In this case, the engaging shaft 2211 can be rotated while being clamped between the boss 2104 and the stopping member 2105. Thus, the engaging shaft 2211 can be rotated stably, instead of falling off.

Moreover, the boss 2104 can be fitted in the engaging opening 2212 when the cover 220 is closed, and when the cover 220 is opened, the boss 2104 will be separated from the engaging opening 2212. Thus, the closed cover 220 is parallel to the intermediate member 210, thereby ensuring a compact and stable structure.

In some embodiments, two engaging openings 2212 may be formed and arranged in a line parallel to the engaging shaft 2211, and accordingly, two bosses 2104 are also provided, so that the engaging shaft 2211 can be rotated more stably while being clamped between the stopping member 2105 and the two bosses 2104.

In some embodiments, as illustrated in FIG. 8, a surface of the boss 2104 facing the stopping member 2105 may have a substantially “S” shape, so that the engaging shaft 2211 can be easily placed into the space defined by the boss 2104 and the stopping member 2105.

In some embodiments, also as illustrated in FIG. 8, the free end of the stopping member 2105 may be perpendicular to the rest of the stopping member 2105. For example, the reset of the stopping member 2105 extends vertically from the first surface 219a of the intermediate member 210, and the free end of the stopping member 2105 is bent and extends horizontally, so that the engaging shaft 2211 can be more stably rotated while by clamped between the boss 2104 and the stopping member 2105.

In the embodiments of the present disclosure, the first snap 2102 and the second snap 2103 have a same structure. Thus, only the structure of the first snap 2102 is described in detail, serving as an example.

Specifically, the first snap 2102 includes a first hook 2106 and a second hook 2107 protruded from the first surface 219a of the intermediate member 202. The first hook 2106 and the second hook 2107 are arranged adjacent to and face away from each other. That is, a hooking portion of the first hook 2106 extends from a main body of the first hook 2106 in a direction facing away from the second hook 2107, and vice versa.

In this case, when the cover 220 is closed to the intermediate member 210, the first hook 2106 and the second hook 2107 of the first snap 2102 will be snapped into the first snapping hole 222 of the cover 220, and hooked on an edge of the first snapping hole 222. Thus, the cover 220 can locked in a closed state. When the cover 220 needs to be opened, the first hook 2106 and the second hook 2107 of the first snap 2102 can be pinched towards each other, so that the first snap 2102 can be separated from the first engaging hole 222, and thus the cover 220 can be opened.

It should be noted that the structure of the second snap 2103 is the same with that of the first snap 2102, and thus the fit between the second snap 2103 and the second snapping hole 310 is similar to that of the first snap 2102 and the first snapping hole 222.

Specifically, a first hook 2108 and a second hook 2109 of the second snap 2103 may be snapped into the second snapping hole 310 of the base 300, so that the base 300 can be connected to the intermediate member 210. Further, the first hook 2108 and the second hook 2109 of the second snap 2103 are hooked on an edge of the second snapping hole 310 of the base 300, and thus the base 300 can be stably connected to the intermediate member 210 and moved along with the intermediate member 210. When the base 300 is in the second position and needs to be detached from the intermediate member 210 during the tissue embedding, the first hook 2108 and the second hook 2109 of the second snap 2103 may be pinched towards each other, so that the second snap 2103 can be separated from the second snapping hole 310, and thus the base 300 can be removed. In this case, the wax block can be formed without the base 300 embedded. Therefore, the base 300 does not need to be cut, while the wax block is sectioned, thereby improving the microtome blade's life and enhancing the sectioning efficiency.

In some embodiments, two first snaps 2102 may be provided at two opposite edges of the through hole 218 of the intermediate member 210, and the first engaging member 2101 is arranged between an edge of the intermediate member 210 and one of the first snaps 2102. Accordingly, two first snapping holes 222 are formed in the cover 220 and correspond to the two first snaps 2102, respectively. That is, the two first snaps 2102 are in a one-to-one correspondence with the two first snapping holes 222, and the two first snapping holes 222 are also arranged at the two opposite edges of the through hole 218 of the intermediate member 210, when the cover 220 is closed to the intermediate member 210. Further, the engaging opening 2212 is arranged between the engaging shaft 2211 and one of the two first snapping holes 222 adjacent to the engaging shaft 2211.

Thus, the cover 220 can be more stably locked to the intermediate member 210.

Furthermore, as illustrated in FIGS. 10 and 11, the cover 220 also includes a grasping member 223 extending from a surface of the cover 220 facing away from the intermediate member 210 when the cover 220 is closed to the intermediate 210. The grasping member 223 is arranged between the two first snapping holes 222, and adjacent to the other one of the two first snapping holes 222, i.e. the first snapping hole 222 away from the engaging shaft 2211, instead of the first snapping hole 222 adjacent to the engaging shaft 2211. A free end of the grasping member 223 is bent towards the first snapping hole 222 away from the engaging shaft 2211, so as to extend substantially parallel to the cover 210.

Thus, a user can grasp the grasping member 223 when he/she opens or closes the cover 220, so that the cover 220 can be opened and closed easily.

In some embodiments, four second snaps 2103 may be provided at the two opposite edges of the through hole 218 of the intermediate member 210, two of the four second snaps 2103 are arranged at one of the two opposite edges of the through hole 218, and the other two of the four second snaps 2103 are arranged at the other one of the two opposite edges of the through hole 218. Accordingly, four second snapping holes 310 are formed in the base 300 and correspond to the four second snaps 2103, respectively. That is, the four second snaps 2103 are in a one-to-one correspondence with the four second snapping holes 310, and thus two of the four second snapping holes 310 are arranged at one of the two opposite edges of the through hole 218, and the other two of the four second snapping holes 310 are arranged at the other one of the two opposite edges of the through hole 218, when the base 300 is connected to the intermediate member 210.

Thus, the base 300 can be connected to the intermediate member 210 stably.

In some embodiments, as illustrated in FIGS. 12 and 13, the base 300 includes a receptacle 320 having an opening 3201 and a flange 330 connected to the receptacle 320 and surrounding the opening 3201 of the receptacle 320, and the second snapping hole 310 is formed in the flange 330 of the base 300. Thus, the base 300 can be stably connected to the intermediate member 210 through the flange 330 of the base 300.

Further, the receptacle 320 includes a bottom 3202 and a side wall 3203 extending from an edge of the bottom 3202, and the bottom 3202 defines a plurality of through holes 3204. Thus, the receptacle 320 defines a cavity therein, for receiving the tissue sample 400. Moreover, reagents can flow through the through holes 3204 in the bottom 3202, so as to facilitate the tissue processing and/or embedding.

In some embodiments, the side wall 3203 may extend vertically from the edge of the bottom 3202, or the side wall 3203 may extend obliquely from the edge of the bottom 3202, which is not specifically limited herein.

In some embodiments, as illustrated in FIGS. 10 and 11, the cover 220 includes a body 224 and a fixing member 225 arranged to a surface of the body 224 configured to be in contact with the intermediate member 210, and the retaining member 230 is fixed to the body 224 by the fixing member 225.

Further, the fixing member 225 includes a plurality of projections 226 extending from the surface of the body 224, and the retaining member 230 is surrounded and fixed by the plurality of projections 226. For example, the retaining member 230 may have a certain elasticity, and a size of the retaining member 230 may be slightly larger than a size of a space surrounded by the plurality of projections 226, so that the retaining member 230 can be inserted into the space surrounded by the plurality of projections 226 and fixed therein. Moreover, the retaining member 230 may be also adhered to the surface of the cover 220. Thus, the retaining member 230 can be stably fixed to the body 224, and hence move along with the cover 220. When the cover 220 is closed after the tissue sample 400 is placed in the base 300, the retaining member 230 together with the plurality of projections 226 will pass through the through hole 218 of the intermediate member 210, so as to retain and oriented the tissue sample 400 in the base 300.

In some embodiments, the body 224 defines a plurality of through holes 227 overlapping at least part of the retaining member 230. Thus, the reagents can flow through the through holes 227 in the body 224 and further through the retaining member 230, so as to facilitate the tissue processing and/or embedding.

Further, the retaining member 230 may be a solid block made of a three-dimensional resilient material, i.e. having a three-dimensional shape (for example, a cube or a cuboid, which is not specifically limited herein), such as foam (including polyether, polyurethane, etc.), hydrogel (including natural/synthetic materials such as agar, gelatin, carbomer, protein gel, alginates, polyvinyl alcohol, polyacrylamide, polyacrylic acid, etc.), organogel, aerogel, and so on. That is, the three-dimensional resilient material may be a block material.

The three-dimensional resilient material of the retaining member 230 allows at least part of the reagents for the tissue processing and embedding to pass through.

It may be understood that the retaining member 230 should have a shape similar to a shape defined by the plurality of projections 226, so that the retaining member 230 can be fixed by the plurality of projections 226 firmly.

Moreover, the retaining member 230 should have a cross section similar to and slightly smaller than that of the through hole 218 of the intermediate member 210, so that the retaining member 230 can pass through the through hole 218 of the intermediate member 210 smoothly.

In some embodiments, as illustrated in FIGS. 6-9 in combination with FIGS. 1-5, the intermediate member 210 includes a rim 215 extending outwards from an outer side wall of the intermediate member 210, the frame 100 includes a first limiting member 110 on an inner side wall of the frame 100 and adjacent to a first edge of the inner side wall of the frame 100, and the first limiting member 110 is configured to be fitted with the rim 215 to limit the retaining assembly 200 in the first position. Thus, the intermediate member 210 and the base 300 can be held in the first position through cooperation between the rim 215 and the first limiting member 110.

In some embodiments, the first limiting member 110 includes a first protrusion 111 extending inwards from the inner side wall of the frame 100, and the first protrusion 111 is configured to support a surface of the rim 215 of the intermediate member 210 facing the base 300 when the retaining assembly 200 is in the first position. Thus, the first protrusion 111 prevents the intermediate member 210 from moving in a direction towards the base 300, and the intermediate member 210 and the base 300 are held in the first position stably.

In some embodiments, the surface of the rim 215 of the intermediate member 210 facing the base 300 defines a first groove 216, and the first protrusion 111 is configured to be fitted in the first groove 216 when the retaining assembly 200 is in the first position. Thus, the intermediate member 210 and the base 300 can be held in the first position more stably.

In some embodiments, the first limiting member 110 further includes a second protrusion 112 extending inwards from the inner side wall of the frame 100, the first protrusion 111 is spaced apart from the second protrusion 112 in a direction from the first position to the second direction, also in a direction from the second position to the first position, i.e. in a moving direction of the intermediate member 210, and the second protrusion 112 is configured to abut against a surface of the rim 215 of the intermediate member 210 facing away from the base 300 when the retaining assembly 200 is in the first position. Thus, the second protrusion 112 prevents the intermediate member 210 from moving in a direction running away from the base 300, and the intermediate member 210 and hence the base 300 can be firmly clamped in the first position by the first protrusion 111 and the second protrusion 112.

In some embodiments, the surface of the rim 215 of the intermediate member 210 facing away from the base 300 defines a second groove 217, and the second protrusion 112 is configured to be fitted in the second groove 217 when the retaining assembly 200 is in the first position. Thus, the intermediate member 210 and the base 300 can be held in the first position more stably.

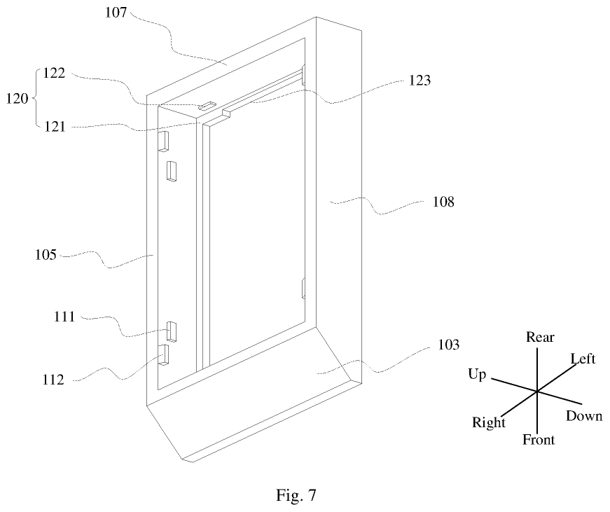

In some embodiments, as illustrated in FIG. 7, the frame 100 include a second limiting member 120 on the inner side wall of the frame 100 and adjacent to a second edge of the inner side wall of the frame 100, the second edge of the inner side wall of the frame 100 is opposite to the first edge of the inner side wall of the frame 100 in the direction from the first position to the second position, also in the direction from the second position to the first position, i.e. in the moving direction of the intermediate member 210, and the second limiting member 120 is configured to be fitted with the rim 215 to limit the retaining assembly 200 in the second position. Thus, the retaining assembly 200 and the base 300 can be held in the second position through cooperation between the rim 215 and the second limiting member 120. Further, the second edge of the inner side wall of the frame 100 is arranged below the first edge of the inner side wall of the frame 100 in the direction from the first position to the second position.

In some embodiments, as illustrated in FIG. 7, the second limiting member 120 includes a step 121 extending inwards from the second edge of the inner side wall of the frame 100, and the step 121 is configured to support the surface of the rim 215 facing the base 300 when the retaining assembly 200 is in the second position. Thus, the step 121 can prevent the intermediate member 210 from moving in the direction towards the base 300, and the intermediate member 210 and the base 300 are held in the second position stably.

In some embodiments, as illustrated in FIG. 7, the second limiting member 120 includes a third protrusion 122 extending inwards from the inner side wall of the frame 100, the step 121 is spaced part from the third protrusion 122 in the direction from the first position to the second direction, also in the direction from the second position to the first position, i.e. in the moving direction of the intermediate member 210, and the third protrusion 122 is configured to abut against the surface of the rim 215 facing away from the base 300 when the retaining assembly 200 is in the second position. Thus, the third protrusion 122 can prevent the intermediate member 210 from moving in the direction running away from the base 300, and the intermediate member 210 and the base 300 can be firmly clamped in the second position by the step 121 and the third protrusion 122.

Furthermore, the first protrusion 111 is arranged below the second protrusion 112 in the direction from the first position to the second position, and the first protrusion 111 and the second protrusion 112 are alternated in a direction perpendicular to the direction from the first position to the second position. Also, the step 121 is arranged below the third protrusion 122 in the direction from the first position to the second position.

In some embodiments, four first protrusions 111 and four second protrusions 112 are provided on two opposite inner side walls of the frame 100. Two first protrusions 111 and two second protrusions 112 are arranged on one of the two opposite inner side walls of the frame 100, and other two first protrusions 111 and other two second protrusions 112 are arranged on the other one of the two opposite inner side walls of the frame 100. The two first protrusions 111 on the same inner side of the frame 100 are spaced in the direction perpendicular to the direction from the first position to the second position, and the two second protrusions on the same inner side wall of the frame 100 are also spaced in the direction perpendicular to the direction from the first position to the second position. The four first protrusions 111 have a same level, and the four second protrusions 112 have a same level. The same level of the four first protrusions 111 is lower than the same level of the four second protrusions 112.

Moreover, the step 121 is arranged around a whole inner peripheral surface of the frame 100 at the second edge of the inner side wall of the frame 100, and four third protrusions 122 are provided on other two opposite inner side walls of the frame 100. Two third protrusions 122 are provided on one of the other two opposite inner side walls of the frame 100, and other two third protrusions 122 are provided on the other one of the other two opposite inner side walls of the frame 100. The four third protrusions 122 have a same level higher than a level of the step 121 in the direction from the first position to the second position.

Specifically, the frame 100 has a substantially rectangular shape, and includes a first side wall 103, a second side wall 105, a third side wall 107 and a fourth side wall 108 coupled end to end in sequence to enclose the accommodating chamber 101. The first and third side walls 103, 107 are arrange oppositely in a front-rear direction, and the second and fourth side walls 105, 108 are arrange oppositely in a left-right direction. The accommodating chamber 101 is opened to the outside in an up-down direction. That is, a top and a bottom of the frame 100 are opened.

The frame 100 is provided with two first protrusions 111 extending from an inner surface of the second side wall 105, and other two first protrusions 111 extending from an inner surface of the fourth side wall 108. The two first protrusions 111 on the second side wall 105 and the two first protrusions 111 on the fourth side wall 108 are oppositely arranged, respectively. The four first protrusions 111 have a same level, and are closer to a top edge of the frame 100 instead of a bottom edge of the frame 100.

The frame 100 is provided with two second protrusions 112 extending from the inner surface of the second side wall 105, and other two second protrusions 112 extending from the inner surface of the fourth side wall 108. The two second protrusions 112 on the second side wall 105 and the two second protrusions 112 on the fourth side wall 108 are oppositely arranged, respectively. The four second protrusions 112 have a same level, and are closer to the top edge of the frame 100 instead of the bottom edge of the frame 100. The four second protrusions 112 have a level higher than the four first protrusions 111 in the up-down direction. In addition, the second protrusions 112 are not aligned with the first protrusions 111 in the up-down direction. That is, the second protrusions 112 are alternated with the first protrusions 111 in the front-rear direction.

The frame 100 is provided with the step 121 extending from an inner peripheral surface of the frame 100 at the bottom edge of the frame 100. That is, the step 121 is formed on the inner surfaces of the first to fourth side walls 103, 105, 107, 108. In addition, two notches 123 are defined in the step 121 at the first and third side walls 107, respectively.

The frame 100 is provided with two third protrusions 122 extending from the inner surface of the first side wall 103, and other two third protrusions 122 extending from the inner surface of the third side wall 107. The two third protrusions 122 on the first side wall 103 and the two third protrusions 122 on the third side wall 107 are oppositely arranged, respectively. The four third protrusions 122 have a same level, and are closer to the bottom edge of the frame 100 instead of the top edge of the frame 100. The four second protrusions 112 have a level higher than the step 121 and lower than the first protrusions 111 in the up-down direction.

The frame 100 is provided with an inclined outer surface at the first side wall 103. A label may be placed on the inclined outer surface to identify the tissue sample 400. The inclined outer surface can be configured to receive a label such that the label clicks into the inclined outer surface of the frame 100. Alternatively, the frame 100 may have a textured surface and be put through an inkjet printing system, such as Leica IPC ink jet printer.

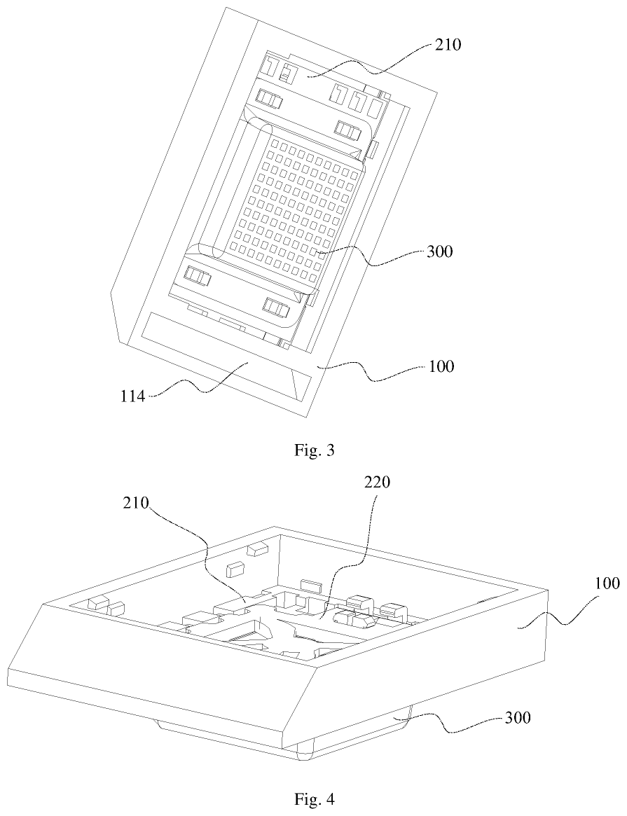

As illustrated in FIG. 3, the frame 100 is further provided with an elongate groove 114 in a bottom surface of the frame 100 at the first side wall 103, and the elongate groove 114 extends in the left-right direction and is used for grasping and handling by the user.

In correspondence to the above configuration of the frame 100, the intermediate member 210 has a substantially rectangular bracket structure, and includes a first bracket segment 211, a second bracket segment 212, a third bracket segment 213 and a fourth bracket segment 214 coupled end to end in sequence to define the through hole 218. The first and third bracket segments 211, 213 are arrange oppositely in the front-rear direction, and the second and fourth bracket segments 212, 214 are arrange oppositely in the left-right direction.

Further, the intermediate member 210 is provided with the rim 215 extending outwards from an outer peripheral surface at a top edge of the intermediate member 210. That is, the rim 215 is formed on outer surfaces of the first to fourth bracket segments 211, 212, 213, 214.

Further, the intermediate member 210 is provided with two first grooves 216 formed in the bottom surface of the rim 215 at the second bracket segment 212, and other two first grooves 216 formed in the bottom surface of the rim 215 at the fourth bracket segment 214. The two first grooves 216 at the second bracket segment 212 and the two first grooves 216 at the fourth bracket segment 214 are oppositely arranged, respectively.

Further, the intermediate member 210 is provided with two second grooves 217 formed in the top surface of the rim 215 at the second bracket segment 212, and other two second grooves 217 formed in the top surface of the rim 215 at the fourth bracket segment 214. The two second grooves 217 at the second bracket segment 212 and the two second grooves 217 at the fourth bracket segment 214 are oppositely arranged, respectively. The second groove 217 is not aligned with the first groove 216 in the up-down direction. That is, the second groove 217 is alternated with the first groove 216 in the front-rear direction.

Moreover, the intermediate member 210 further includes a plurality of holes 2111 in the first bracket segment 211, and the plurality of holes 2111 are adjacent to the rim 215.

The cassette 1000 according to the embodiments of the present disclosure may have a following operation process.

When the retaining assembly 200 is in the first position, the cover 220 is opened, and the tissue sample 400 is placed in the base 300. Then, the cover 220 is closed, and the retaining member 230 holds and orients the tissue sample 400 in the base 300. Further, the tissue sample 400 is processed by a serious of procedures, such as clearing and dehydration. For example, a dehydration reagent is applied to pass through the cover 220, the retaining member 230, the tissue sample 400, and the base 300 in turn. It should be noted that the dehydration reagent may also pass through the tissue sample 400 in other directions, and this is not limited herein. After the processing of the tissue sample 400, the intermediate member 210 and the base 300 are moved from the first position to the second position for embedding the tissue sample 400. Then, a paraffin wax is injected to pass through the cover 220, the retaining member 230, the tissue sample 400, and the base 300 in turn. After the injection and solidification of the paraffin wax, the base 300 is detached from the intermediate member 210, so that the wax block without the base 300 can be obtained. Then, the wax block carried with the tissue sample 400 can be further sectioned, without cutting the base 300.

In the cassette 1000 according to embodiments of the present disclosure, since the base 300 can be removed during the tissue embedding, the wax block without the base 300 can be obtained, and thus the wax block including the tissue sample 400 can be sectioned, without cutting the base 300. Therefore, the service life of the microtome blade can be improved, and the sectioning efficiency can be enhanced.

Reference throughout this specification to “an embodiment.” “some embodiments,” “one embodiment”, “another example,” “an example,” “a specific examples,” or “some examples,” means that a particular feature, structure, material, or characteristic described in connection with the embodiment or example is included in at least one embodiment or example of the present disclosure. Thus, the appearances of the phrases such as “in some embodiments.” “in one embodiment”, “in an embodiment”, “in another example, “in an example,” “in a specific examples,” or “in some examples,” in various places throughout this specification are not necessarily referring to the same embodiment or example of the present disclosure. Furthermore, the particular features, structures, materials, or characteristics may be combined in any suitable manner in one or more embodiments or examples.

Although explanatory embodiments have been shown and described, it would be appreciated by those skilled in the art that the above embodiments cannot be construed to limit the present disclosure, and changes, alternatives, and modifications can be made in the embodiments without departing from spirit, principles and scope of the present disclosure.

1. A cassette for holding and orientating a tissue sample, comprising:
a frame defining an accommodating chamber;
a retaining assembly coupled to the frame and movably arranged in the accommodating chamber; and
a base detachably coupled to the retaining assembly for receiving the tissue sample and configured to be moved along with the retaining assembly, the retaining assembly being configured to retain the tissue sample in the base,
wherein the retaining assembly and the base are movable between a first position where the base is received in the accommodating chamber and a second position where the base is protruded out of the accommodating chamber, and the base is configured to be detached from the retaining assembly when the retaining assembly is in the second position.

2. The cassette according to claim 1, wherein the retaining assembly comprises:
an intermediate member arranged in the accommodating chamber and having a through hole in the middle, the intermediate member being movable between the first position and the second position;
a cover rotatably connected to the intermediate member and configured to be closed to cover the through hole; and
a retaining member connected to the cover and configured to pass through the through hole to retain the tissue sample in the base.

3. The cassette according to claim 2, wherein the base is detachably connected to the intermediate member and configured to be moved along with the intermediate member between the first position and the second position.

4. The cassette according to claim 3, wherein the intermediate member has a first surface and a second surface facing away from each other, the cover is rotatably connected to the first surface, and the base is detachably connected to the second surface.

5. The cassette according to claim 4, wherein the intermediate member comprises a first engaging member and a first snap protruded from the first surface, and a second snap protruded from the second surface;
the cover has a second engaging member rotatably engaged with the first engaging member, and a first snapping hole into which the first snap is configured to be snapped; and
the base has a second snapping hole into which the second snap is configured to be snapped.

6. The cassette according to claim 5, wherein two first snaps are provided at two opposite edges of the through hole of the intermediate member, and the first engaging member is arranged between an edge of the intermediate member and one of the first snaps; and
two first snapping holes are formed in the cover and correspond to the two first snaps, respectively.

7. The cassette according to claim 5, wherein four second snaps are provided at two opposite edges of the through hole of the intermediate member, two of the four second snaps are arranged at one of the two opposite edges of the through hole, and the other two of the four second snaps are arranged at the other one of the two opposite edges of the through hole; and
four second snapping holes are formed in the base and correspond to the four second snaps, respectively.

8. The cassette according to claim 5, wherein the base comprises a receptacle having an opening and a flange connected to the receptacle and surrounding the opening of the receptacle, and the second snapping hole is formed in the flange of the base.

9. The cassette according to claim 8, wherein the receptacle comprises a bottom and a side wall extending from an edge of the bottom, and the bottom defines a plurality of through holes.

10. The cassette according to claim 2, wherein the cover comprises a body and a fixing member arranged to a surface of the body configured to be in contact with the intermediate member, and the retaining member is fixed to the body by the fixing member.

11. The cassette according to claim 10, wherein the fixing member comprises a plurality of projections extending from the surface of the body, and the retaining member is surrounded and fixed by the plurality of projections.

12. The cassette according to claim 10, wherein the body defines a plurality of through holes overlapping at least part of the retaining member.

13. The cassette according to claim 2, wherein the intermediate member comprises a rim extending outwards from an outer side wall of the intermediate member, the frame comprises a first limiting member on an inner side wall of the frame and adjacent to a first edge of the inner side wall of the frame, and the first limiting member is configured to be fitted with the rim to limit the retaining assembly in the first position.

14. The cassette according to claim 13, wherein the first limiting member comprises a first protrusion extending inwards from the inner side wall of the frame, and the first protrusion is configured to support a surface of the rim of the intermediate member facing the base when the retaining assembly is in the first position.

15. The cassette according to claim 14, wherein the surface of the rim of the intermediate member facing the base defines a first groove, and the first protrusion is configured to be fitted in the first groove when the retaining assembly is in the first position.

16. The cassette according to claim 13, wherein the first limiting member further comprises a second protrusion extending inwards from the inner side wall of the frame, the first protrusion is spaced apart from the second protrusion in a direction from the first position to the second direction, and the second protrusion is configured to abut against a surface of the rim of the intermediate member facing away from the base when the retaining assembly is in the first position.

17. The cassette according to claim 16, wherein the surface of the rim of the intermediate member facing away from the base defines a second groove, and the second protrusion is configured to be fitted in the second groove when the retaining assembly is in the first position.

18. The cassette according to claim 13, wherein the frame comprises a second limiting member on the inner side wall of the frame and adjacent to a second edge of the inner side wall of the frame, the second edge of the inner side wall of the frame is opposite to the first edge of the inner side wall of the frame in a direction from the first position to the second position, and the second limiting member is configured to be fitted with the rim to limit the retaining assembly in the second position.

19. The cassette according to claim 18, wherein the second limiting member comprises a step extending inwards from the second edge of the inner side wall of the frame, and the step is configured to support a surface of the rim facing the base when the retaining assembly is in the second position.

20. The cassette according to claim 19, wherein the second limiting member comprises a third protrusion extending inwards from the inner side wall of the frame, the step is spaced part from the third protrusion in the direction from the first position to the second direction, and the third protrusion is configured to abut against a surface of the rim facing away from the base when the retaining assembly is in the second position.
